(19) United States
(12) Patent Application Publication
(10) Pub. No.: US 2008/0195038 A1
(43) Pub. Date: Aug. 14, 2008

(54) Device for Supplying Fluid to an Ear

(76) Inventors: Michael James Hill, Hale (GB); Mark Llewellyn, Cheshire (GB)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/572,211

(22) PCT Filed: May 17, 2005
(86) PCT No.: PCT/GB2005/001904

§ 371 Date: Feb. 5, 2008

(30) Foreign Application Priority Data
Jul. 15, 2004 (GB) 0415808.5

(51) Int. Cl. A61M 11/06; A61M 37/00

(52) U.S. Cl. 604/73; 128/200.23; 604/181

(57) ABSTRACT

Device (10) for dispensing a pre-defined quantity of oil to an ear, comprising a nozzle (20) operably coupled to the vessel (12) containing oil (14), the nozzle (20) being adapted to be placed in the ear and capable of expelling the oil (14) from the vessel (12) when desired and permitting expulsion when the vessel (12) and/or device (10) is in a substantially upright or substantially inverted position. The present application also relates to a kit of parts for producing the device (10).

The present invention relates to a device for dispensing a pre-defined quantity of fluid to an ear which is both simple in construction and easy to use.

A number of medical conditions are associated with that of the ear and whilst most conditions can be easily treated, frequently the treatment requires a fluid to be placed in the ear and this can be problematic, not least because when a person is upright, the fluid may drain from the ear. Commonly, the ears are treated by means of a medicament in the form of a fluid which can be dispensed to the ear with a device which dispenses the fluid in a drop wise manner. A device which is commonly used comprises a vial with a screw lid containing the medicament and the lid being adapted such that it also incorporates a pipette with a rubber nipple, which can then be used to dispense a quantity of the medicament into the ear. Usually a procedure using the device requires another person to accurately drop the medicament into the ear whilst a patient is lying on their side. Therefore, treatment of both ears can be problematic, as when the patient turns their head so that their other ear is presented in an upright position, the medicament may flow from the ear that was originally treated. A tissue or bung is usually required to maintain the fluid in the ear such that the medicament may be retained for an effective amount of time. Should a patient treat themselves, spillages can also result as it can be quite difficult to treat an area which is not visible. Furthermore, due to the utilitarian design of the pipettes, it can be most difficult to control the quantity of medicament being dispensed and this can be most problematic should a certain does of medicament be required. Cleaning ears with ear buds can also cause problems with wax build up as the buds can compact rather than remove the wax and it is therefore preferable to use a method of wax removal that also alleviates this problem.

A number of devices have been proposed in order to address one or more of the problems associated with the commonly used device. For example, U.S. Pat. No. 4,995,867 discloses a aural medication administration device with allows a patient to self-administer aural medication to the ear disclosed in any plane. U.S. Pat. No. 6,645,173 further discloses a medication dispensing device which can be attached to the head of an individual by a head band having a medicinal depositary reservoir located at the top of the head band, wherein a measured amount of liquid medication would be inserted therein and the device would hold the medication until released and dispensed into the ear canal through an ear piece. However, both devices are cumbersome in their use and require the medicaments to be inserted into each device prior to dispensing the medicament.

It is therefore an object of the present invention to alleviate one or more of the associated problems with prior art devices and it is also to provide for a device which is capable of dispensing an accurate and pre-defined quantity of fluid to an ear which is simple in its construction and effectively expels a known quantity of fluid into the ear in a procedure which can be easily performed by a patient on their own.

In accordance with the present invention, there is provided a device for dispensing a pre-defined quantity of oil to an ear, comprising a nozzle operably coupled to a vessel containing oil, the nozzle being adapted to be placed in the ear and capable of expelling the oil from the vessel when desired and permitting expulsion when the vessel and/or device is in a substantially upright or substantially inverted position.

Therefore the present invention provides a device that may be used for dispensing any oil to the ear so as to treat a medical condition where the oil is encapsulated and protected within the device at all times until actuation of the metering valve when the oil is expelled from the nozzle. If required, the vessel may be resiliently deformable, so that an individual can dispense the oil by simply squeezing the sides of the vessel so as to force the oil from the nozzle. The device may further permit the correct unit dose of an oil to be dispensed to the ear and it will be apparent to one skilled in the art that this will be important with regard to medicaments that require a given quantity of therapeutic agent for it's effective action.

Any oil may be used in accordance with the present invention. Preferably, an oil is a fixed oil, which also includes fatty oils. Such oils commonly occur in nature and are derived from plants and comprise esters of glycerin and fatty acids. Any oil such a vegetable or nut oil (including mixtures thereof) may be used in accordance with the present invention, however, constituent(s) and/or derivative(s) of an oil may also be used. It should be understood that vegetable or nut oil is any oil or derivative that can be obtained from plants.

A substantially upright or substantially inverted position of the vessel should be construed as a position within ±30° of the vertical axis of the vessel.

As the nozzle is adapted to be received in the ear (auditory canal), the device can be accommodated in the ear with comfort and it also ensures that the required therapeutic quantity of oil is placed in the desired part of the ear (such as just before the ear drum within the auditory canal). Preferably, the nozzle is disposed at an angle perpendicular to the vessel. The nozzle may be connected to a button so as to actuate the expulsion of the oil. It will be apparent to one skilled in the art that the expulsion of the oil from the vessel may be actuated in a number of different ways such as the oil may be held in the vessel under pressure and the oil may be expelled by the release of a pressurized valve within the vessel and the valve in turn permitting a pre-determine quantity of oil to be released from the vessel in a similar manner to a asthma inhaler which provides a metered dose from an aerosol canister. The oil may also be expelled from the vessel by employing a propellant in addition to the oil and it will be apparent that the use of a propellant may also be in conjunction with a pressurized valve. Alternatively, the oil may be expelled by means of a pump action mechanism. The oil may be expelled from the nozzle as one or more droplets, each drop having a known volume. Alternatively, the known volume of oil may be atomized from the nozzle so as to assist the action of the oil in ear.

It will be apparent that the device may be used for a number of uses and most common medical conditions of the ear stem from excess ear wax production which may be treated by a wax softening or dissolving agent. The device could also be utilized to supply oil in order to reduce or eradicate ingress of water (of any type) in the ear. The oil may also be used to as a lubricant to any part of the ear and in particular, the outer ear and furthermore, the oil may be used to lubricate the ear, so as to facilitate the insertion of a medical device. The oil could also be deployed so to alleviate any discomfort associated with external dynamic or static ambient pressure changes, for example on aero plane flights etc.

The oil may comprise a vegetable/nut oil or mixtures thereof. The oil may alternatively or additionally comprise a mineral oil, or mixtures thereof. The use of a mineral oil may be to act as a thinner for a vegetable/nut oil if required. Preferably, a mineral oil is one that can be distilled down into a white mineral or petroleum oil or paraffin oil. More preferably, the mineral oil is Paraffinum Liquidum. Should a vegetable oil be used to soften or dissolve the ear wax, it will preferably be olive oil and this may be an oil approved by the relevant national or international agencies for use in the human and/or animal bodies. The oil may also comprise or additionally comprise oils derived from a range of organisms, such as animals and fish. A suitable vegetable oil, may comprise Tea Tree oil. The oil may also contain a pharmaceutically active ingredient and the oil may additionally comprise a pharmaceutically acceptable carrier, or the oil may act as such a carrier. An additive may also be provided with the oil to prevent oxidation and/or microbial contamination and such an additive may be a preservative, a n anti-oxidant or an anti-microbial agent for example. The oil may be replaced with an alternative viscous liquid that may be used to soften or dissolve ear wax.

The nozzle may further comprise a rim that it goes around part of the nozzle so as to prevent the oil from leaving the ear during and/or after expulsion from the vessel. The nozzle may be manufactured from a number of materials but would be preferably manufactured from a plastic or rubber material. The device may be sealed so as to prevent tampering and an anti-tampering seal may also be provided so as to indicate whether or not tampering has occurred with the device. Preferably, the device dispenses a metered unit dose of the oil which relates to the quantity required for the successful treatment of an ear condition.

In accordance with a further aspect of the present invention, there is provided a kit of parts for the production of a device for dispensing a pre-defined quantity of oil to an ear, the kit comprising;

(a) a vessel for holing a quantity of oil;

(b) a nozzle adapted to be operably coupled to the and to permit expulsion of the oil when required; and

(c) a quantity of oil.

The kit of parts may be used in order to produce a device as herein above described.

The present invention will now be more fully described by way of example only with reference to the following figure.

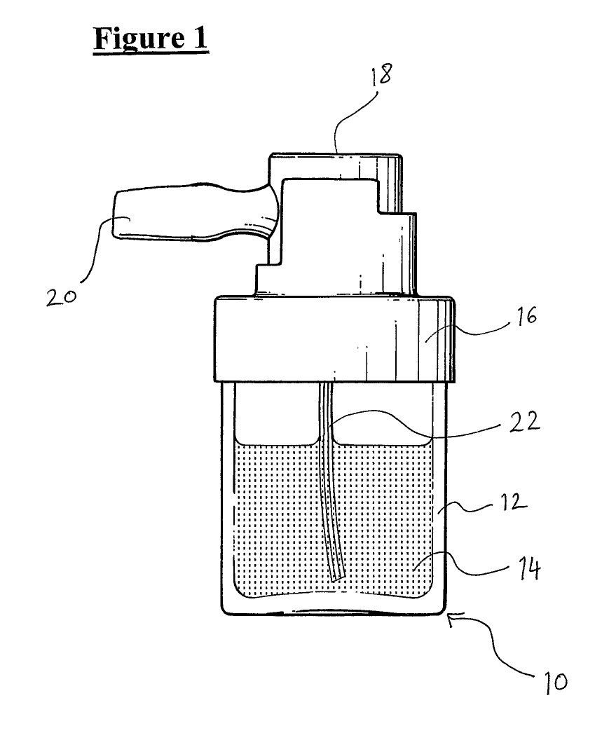

FIG. 1 shows a device in accordance with the present invention.

With reference to FIG. 1, there is provided a device 10 for dispensing an ear wax softening agent to the ear, the device having a vessel 12 containing olive oil 14. The vessel 12 has a cap 16 that seals the vessel and incorporates a pump-action mechanism for expelling the oil 14 and the mechanism is actuated by button 18 which in turn is attached to nozzle 20. An aperture (not shown) runs through the nozzle 20 and permits the oil 14 from being expelled from the device 10. The pump-action mechanism (not shown) includes a capillary tube 22 through which the oil 14 can be expelled from the vessel 12 through the nozzle 20. In the case of a device manufactured for actuation whereby the vessel is inverted, the capillary tube 22 is not required.

In use, the vessel 10 will be manufactured as a sealed device containing a quantity of olive oil 14 for dispensing to the ear so as to soften and/or dissolve the ear wax prior to or in addition to syringing the ear with water or a saline solution. The device 10 can be used in an upright position and by the patient themselves to dispense the oil as required. The pump action mechanism draws the oil 14 up the capillary tube 22 when the button 18 is depressed which expels a desired quantity of oil to the ear via the nozzle 20 which is adapted to fit snugly within the ear. The nozzle 20 may also have a disc disposed around its circumference so as to ensure that the oil 14 does not exit the ear during its application. The device can be made out of a number of materials, but commonly the vessel 12 will be produced from a plastics or glass material with the lid 16 being sealed onto it. The device 10 may also have a label disposed thereon to provide patient information such as dosing etc.

The device 10 can also be adapted for dispensing pharmaceutical agents and if appropriate, the pump action mechanism may be replaced by a pressure valve or atomizer should it be required. In such instances, a capillary tube within the vessel may not be required. Alternatively, the vessel 12 may be deformable and upon deformation of the vessel 12 either by hand or by mechanical means, oil may be expelled.

1. A device for dispensing a pre-defined quantity of oil to an ear, comprising a nozzle operably coupled to a vessel containing oil, the nozzle being adapted to be placed in the ear and capable of expelling, the oil from the vessel when desired and permitting expulsion when the vessel and/or device is in a substantially upright or substantially inverted position and wherein the oil comprises a vegetable oil or mixture of two or more vegetable oils.

2. A device as claimed in claim 1 wherein the nozzle is connected to a button to actuate the expulsion of the oil from the vessel.

3. A device as claimed in claim 1 wherein the oil is expelled by means of a pump action mechanism.

4. A device as claimed in claim 1, wherein the oil is held in the vessel under pressure.

5. A device as claimed in claim 4, wherein the oil is expelled by releasing a pressurized valve in the vessel.

6. A device as claimed in claim 1, wherein the vessel is resiliently deformable.

7. A device as claimed in claim 1, wherein the oil is expelled from a nozzle as one or more droplets of a known volume.

8. A device as claimed in claim 1, wherein a known volume of oil is atomized from the nozzle.

9. A device as claimed in claim 1, wherein the nozzle is disposed at an angle perpendicular to the vessel.

10. A device as claimed in claim 1, wherein the oil is used to soften or dissolve ear wax.

11. A device as claimed in claim 1, wherein the oil is used to reduce or eradicate ingress of water in the ear.

12. A device as claimed in claim 1, wherein the oil is used to lubricate any part of the ear.

13. A device as claimed in claim 12, wherein the oil is used to lubricate the ear, so as to facilitate the insertion of a medical device.

14. A device as claimed in claim 1, wherein the oil is used alleviate discomfort associated with external dynamic or static ambient pressure changes.

15. A device as claimed in claim 1, wherein the oil further comprises an animal and/or fish oil or a mixture of two or more animal and/or fish oils.

16. A device as claimed in claim 1, wherein the oil further comprises a mineral oil or mixture of two or more mineral oils.

17. A device as claimed in claim 1, wherein the vegetable oil comprises olive oil.

18. A device as claimed in claim 1, wherein the vegetable oil comprises Tea Tree oil.

19. A device as claimed in claim 1, wherein the oil further comprises a pharmaceutically active ingredient and/or a pharmaceutically acceptable carrier.

20. A device as claimed in claim 1, wherein an additive is provided with the oil to prevent oxidation and/or microbial contamination.

21. A device as claimed in claim 1, wherein the nozzle further comprises a rim that extends around the nozzle so as to prevent the oil from leaving the ear during and/or after expulsion from the vessel.

22. A device as claimed in claim 1, wherein the nozzle is manufactured from a plastics or rubber material.

23. A device as claimed in claim 1, wherein the device is sealed to prevent tampering.

24. A device as claimed in claim 1, wherein the device dispenses a metered dose of oil.

25. A device as claimed in claim 1, wherein the pre-defined quantity of oil is expelled through a metering valve.

26. A kit of parts for the production of a device for dispensing a predefined quantity of oil to an ear, the kit comprising;
(a) a vessel for holding a quantity of oil;
(b) a nozzle adapted to be operably coupled to the and to permit expulsion of the oil when required; and
(c) a quantity of oil.

27. A kit of parts as claimed in claim 26, wherein the kit is used to produce a device as claimed in claim 1.

28-30. (canceled)
